(19) United States
(12) Patent Application Publication
(10) Pub. No.: US 2022/0267368 A1
(43) Pub. Date: Aug. 25, 2022

(54) METHOD FOR PREPARING BOTULINUM TOXIN

(71) Applicant: PROTOX INC., Hwaseong-si (KR)

(72) Inventors: Chi Jong SONG, Busan (KR); Eun Young LEE, Uiwang-si (KR); Jin Hee AHN, Seoul (KR)

(21) Appl. No.: 17/634,874

(22) PCT Filed: Aug. 14, 2020
(86) PCT No.: PCT/KR2020/010885

§ 371 Date: Feb. 11, 2022

(30) Foreign Application Priority Data
Aug. 14, 2019 (KR) 10-2019-0099531

(51) Int. Cl. C07K 1/18; C07K 1/30; C07K 1/34; C07K 14/33

(52) U.S. Cl. CPC C07K 1/18; C07K 1/30; C07K 1/34; C07K 14/33

(57) ABSTRACT

The present invention relates to a botulinum toxin preparation method capable of obtaining botulinum toxin in a high yield through a simplified process that does not include animal-derived ingredients. The botulinum toxin preparation method according to the present invention does not use any animal ingredients in the overall process, including the culturing of a Clostridium botulinum strain, thereby providing excellent safety, omits a separate nucleic acid removal step using an additive treatment when compared with a conventional isolation process, and performs processing using only ion exchange chromatography, and it was confirmed that the botulinum toxin can be isolated at a remarkably improved yield through a simplified process by using the same buffer and only adjusting the concentration and pH of the buffer, and thus the present invention is a very economical and efficient isolation method so that the botulinum toxin isolated thereby is expected to be effectively used in beauty and medicine fields.

### TECHNICAL FIELD

The present invention relates to a method for preparing botulinum toxin, which is capable of obtaining botulinum toxin with high yield through a simplified process while not including an animal-derived component.

### BACKGROUND ART

Various strains of the genus Clostridium that secrete a neurotoxic toxin have been discovered since the 1890s, and characterization of the toxin secreted by these strains has been identified. The neurotoxic botulinum toxin, which is derived from the strains of the genus Clostridium, is a neurotoxin produced by the growth of Clostridium botulinum in food that has not been properly sterilized or stored in cans that have not been properly sterilized and causes food poisoning, vomiting, visual impairment, motor disturbances, and the like. When this toxin is ingested, the incubation period is 12 to 72 hours, and it prevents the release of the neurotransmitter acetylcholine at a neuromuscular junction, causing muscle paralysis.

Botulinum toxin is a neurotoxic protein consisting of amino acids and is classified into a total of seven types including A, B, C (C1, C2), D, E, F, and G according to a serological characteristic. Each toxin has a toxic protein of about 150 kDa and naturally consists of a complex in which the toxic protein is bound to several non-toxic proteins. A medium complex (300 kDa) consists of a toxic protein and a non-toxic non-hemagglutinin protein, and a large complex (450 kDa) and a large-large complex (900 kDa) are in the form in which the medium complex is bound to hemagglutinin. These non-toxic non-hemagglutinin proteins are known to function to protect a toxin from low pH and various types of proteases in the intestine. Botulinum toxin is first synthesized as a single molecule with a size of 150 kDa and then truncated into a light chain protein of about 50 kDa and a heavy chain protein of about 100 kDa, and the light chain protein and the heavy chain protein are linked again via a disulfide bond to finally form an active botulinum toxin.

Botulinum toxin suppresses the release of the neurotransmitter acetylcholine at the presynaptic cell of the neuromuscular junction. Acetylcholine is present in the synaptic vesicle inside the presynaptic cell, and when an action potential signal arrives at the presynaptic cell, the synaptic vesicle is fused with the presynaptic membrane, and thus acetylcholine is released into a synaptic cleft. SNARE proteins are essential for the fusion of synaptic vesicles and presynaptic membranes and are largely divided into vesicle SNARE (v-SNARE) proteins located at synaptic vesicles and target SNARE (t-SNARE) proteins located at presynaptic membranes. Specifically, synaptobrevin proteins function as v-SNARE, and SNAP-25 and syntaxin proteins function as t-SNARE. Botulinum toxin enters the inside of the presynaptic cell, cleaves SNARE proteins, and thus the proteins no longer function. Therefore, acetylcholine is not released at the presynaptic cell of the neuromuscular junction, and muscle control by nerves becomes impossible, leading to flaccid paralysis. Specifically, the heavy chain of botulinum toxin proteins allows the toxin to enter the inside of the presynaptic cell, and the light chain thereof allows the toxin to cleave SNARE proteins. The seven types of botulinum toxin, including A, B, C (C1, C2), D, E, F, and G, are known to cleave different SNARE proteins.

Since the botulinum toxin is lethal to the human body in a small amount and is easy to mass-produce, it can be used as one of the four major biological weapons along with Bacillus anthracis, Yersinia pestis, and smallpox virus. However, it was found that the systemic injection of type-A botulinum toxin at a dose below a dose that does not affect the human body can paralyze the local muscle at the injection site. Due to this characteristic, it can be widely used as a wrinkle remover, a therapeutic agent for spastic hemiplegia and cerebral palsy, and the like, and as medical indication is increasing, its demand is rapidly increasing. In response to this demand, research on a botulinum toxin production method is being actively conducted.

In this regard, conventionally, various attempts to obtain botulinum toxin with high yield have been made by changing and adding process steps and conditions. For example, U.S. Registered Pat. No. 6,818,409 discloses a method for purifying botulinum toxin using cation-exchange chromatography and lactose gel column chromatography, and U.S. Registered Pat. No. 8,927,229 discloses a method for obtaining botulinum toxin using anion-cation-hydrophobic interaction chromatography while not using an animal-derived component. However, these conventional methods have a problem in that a purification process for obtaining botulinum toxin with high yield is complicated and difficult.

Accordingly, the inventors of the present invention have developed a method capable of obtaining a high-purity toxic protein with high yield by further simplifying the conventional botulinum toxin preparation process without adding/changing a complicated process, and the present invention has been completed based on the facts.

### DISCLOSURE

### Technical Problem

The inventors of the present invention have studied a method for efficiently isolating botulinum toxin with high yield through a more simplified process while not including an animal-derived component and thus established an optimum isolation process according to the present invention, thereby completing the present invention.

The present invention is directed to providing a method for preparing botulinum toxin, which includes: the steps of:
(a) culturing Clostridium botulinum in a culture medium free of animal-derived components to produce botulinum toxin;
(b) acid-precipitating a liquid culture containing the botulinum toxin produced therein;
(c) adding a buffer to the botulinum toxin-containing precipitate resulting from the step (b) to obtain a supernatant, adding ammonium sulfate to obtain a precipitation supernatant, and performing ultrafiltration;
(d) performing primary anion-exchange chromatography to obtain purified botulinum toxin;
(e) adding ammonium sulfate to the purified botulinum toxin resulting from the step (d) to obtain a precipitation supernatant and performing ultrafiltration;
(f) performing secondary anion-exchange chromatography to obtain purified botulinum toxin; and
(g) performing cation-exchange chromatography to concentrate botulinum toxin.

However, technical problems to be solved in the present invention are not limited to the above-described problems, and other problems which are not described herein will be fully understood by those of ordinary skill in the art from the following descriptions.

### Technical Solution

One aspect of the present invention provides a method for preparing botulinum toxin, which includes: the steps of:
(a) culturing Clostridium botulinum in a culture medium free of animal-derived components to produce botulinum toxin;
(b) acid-precipitating a liquid culture containing the botulinum toxin produced therein;
(c) adding a buffer to the botulinum toxin-containing precipitate resulting from the step (b) to obtain a supernatant, adding ammonium sulfate to obtain a precipitation supernatant, and performing ultrafiltration;
(d) performing primary anion-exchange chromatography to obtain purified botulinum toxin;
(e) adding ammonium sulfate to the purified botulinum toxin resulting from the step (d) to obtain a precipitation supernatant and performing ultrafiltration;
(f) performing secondary anion-exchange chromatography to obtain purified botulinum toxin; and
(g) performing cation-exchange chromatography to concentrate botulinum toxin.

According to one embodiment of the present invention, the culture medium may contain phytone peptone, a yeast extract, and glucose.

According to another embodiment of the present invention, the acid precipitation in the step (b) may be performed by adding sulfuric acid or hydrochloric acid so that a pH becomes pH 3.0 to pH 4.5.

According to still another embodiment of the present invention, the buffer in the step (c) may be sodium citrate with pH 4.5 to pH 6.5.

According to yet another embodiment of the present invention, a separate nucleic acid removal process may be omitted before the addition of ammonium sulfate in the step (c).

According to yet another embodiment of the present invention, the ammonium sulfate in the step (c) may be added so that a concentration becomes 40% to 80%(w/v).

According to yet another embodiment of the present invention, the primary anion-exchange chromatography may be performed using a diethylaminoethyl (DEAE)-Sepharose column.

According to yet another embodiment of the present invention, the DEAE-column may have a packing volume of 150 mL to 250 mL.

According to yet another embodiment of the present invention, the ammonium sulfate in the step (e) may be added so that a concentration becomes 30% to 50%(w/v).

According to yet another embodiment of the present invention, the secondary anion-exchange chromatography may be performed using a Q-Sepharose column.

According to yet another embodiment of the present invention, the botulinum toxin in the step (f) may be obtained as a botulinum toxin-containing fraction from a flow through (FT) eluted from anion-exchange chromatography.

According to yet another embodiment of the present invention, the cation-exchange chromatography may be performed using a HS-column

According to yet another embodiment of the present invention, the chromatography processes in the steps (d), (f), and (g) may be performed using the same sodium citrate buffer with pH 4.5 to pH 6.5.

### Advantageous Effects

A method for preparing botulinum toxin according to the present invention does not use any animal components in the overall process, including the culturing of a Clostridium botulinum strain, thereby providing excellent safety, omits a separate nucleic acid removal process through an additive treatment as compared with a conventional isolation process, and performs processes only using ion-exchange chromatography where the same buffer is used, and thus it was confirmed that the botulinum toxin can be isolated with significantly improved yield through a simplified process by only adjusting the concentration and pH of the buffer. Therefore, the method is a very economical and efficient isolation method, and the botulinum toxin isolated thereby is expected to be usefully used in beauty and medicine fields.

### DESCRIPTION OF DRAWINGS

FIG. 1 is a step-by-step view of a basic botulinum toxin preparation process (Process 1) of the present invention.

FIG. 2A is a step-by-step view of a process (Process 2) in which vegetable medium component and chromatography column volume conditions are changed from the process (Process 1) of FIG. 1.

FIG. 2B shows a result obtained by measuring the total amount (mg) and concentration (mg/mL) of respective proteins isolated through Processes 1 and 2.

FIG. 2C shows an SDS-PAGE result of respective purified liquids isolated through Processes 1 and 2.

FIG. 2D shows a result obtained by measuring the toxicity of a culture supernatant (culture) and a final purified liquid (final) isolated through Processes 1 and 2.

FIG. 3A is a step-by-step view of a process (Process 3) modified from the process (Process 2) of FIG. 2A, in which a nucleic acid removal process through protamine sulfate treatment is omitted, a DEAE-Sepharose column volume condition in primary anion-exchange chromatography is changed, and cation-exchange chromatography using a HS-column is added.

FIG. 3B shows a result obtained by measuring the nucleic acid removal efficiency (#1, #2, #3) before and after protamine sulfate treatment and the nucleic acid removal efficiency (#4, #5, #6) before and after treatment with a DEAE-Sepharose column after the packing volume of the DEAE-Sepharose column is changed from 30 mL to 200 mL.

FIG. 3C shows a result obtained by measuring the total amount (mg), concentration (mg/mL), and toxicity of proteins in the final purified liquid in an existing process before addition of a HS-column (#1, #2, #3) and a process (Process 3) changed by adding a HS-column purification process (#4, #5, #6).

FIG. 3D show a result illustrating the nucleic acid removal effect of a purified liquid (#1, #2, #3) finally purified through a Q-column after removal using protamine sulfate and the nucleic acid removal effect of a purified liquid (#4, #5, #6) finally purified through a HS-column after treatment with a DEAE-Sepharose column.

FIG. 4A is a step-by-step view of a finally established process (Process 4) modified from the process (Process 3) of FIG. 3, in which a Q-Sepharose column process in secondary anion-exchange chromatography is modified, and a buffer used in a HS-column process of cation-exchange chromatography is altered to be the same as that used in the Q-Sepharose column process.

FIG. 4B shows a result obtained by measuring the total amount (total protein) and concentration (protein quantity) of respective proteins isolated through Process 3 (#4, #5, #6) and Process 4 (#7).

FIG. 4C shows a result obtained by measuring the toxicity of respective final purified liquids isolated through Process 3 (#4, #5, #6) and Process 4 (#7.

FIG. 4D shows an SDS-PAGE result of respective purified liquids obtained after Q-column purification and after HS-column purification through Process 3 and Process 4.

### MODES OF THE INVENTION

The inventors of the present invention have studied a method for efficiently isolating botulinum toxin with high yield through a more simplified process while not including an animal-derived component and thus established an optimum botulinum toxin isolation process according to the present invention, thereby completing the present invention.

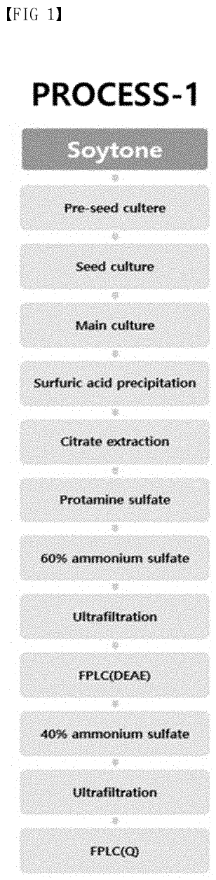

The inventors of the present invention have established a final process according to the present invention by deleting, adding, and/or changing some processes in the conventional botulinum toxin preparation process (Process 1) shown in FIG. 1 through examples.

According to one embodiment of the present invention, a process in which vegetable medium component and Q-Sepharose column volume conditions are changed from the process shown in FIG. 1 is developed, and as a result of comparing the concentration of proteins isolated by the two processes and the purity and toxicity of purified fractions, it was confirmed that the recovery rate of botulinum toxin was increased according to changed conditions (see Example 2).

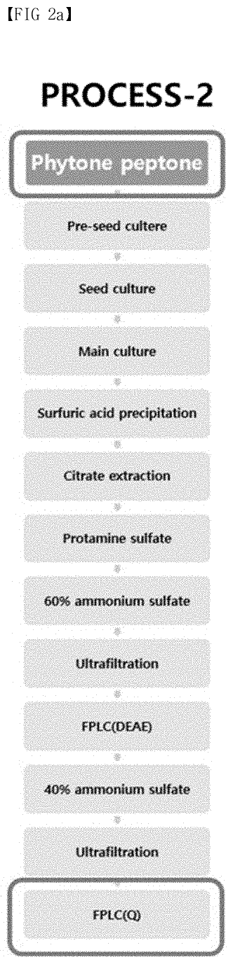

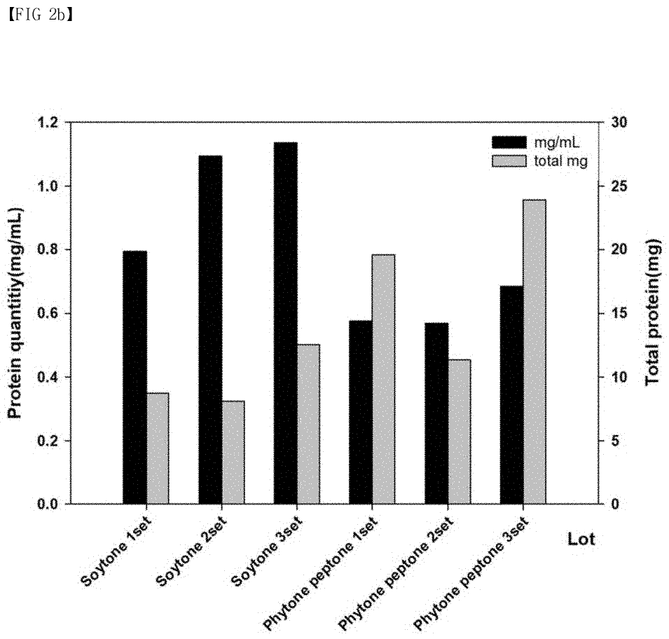

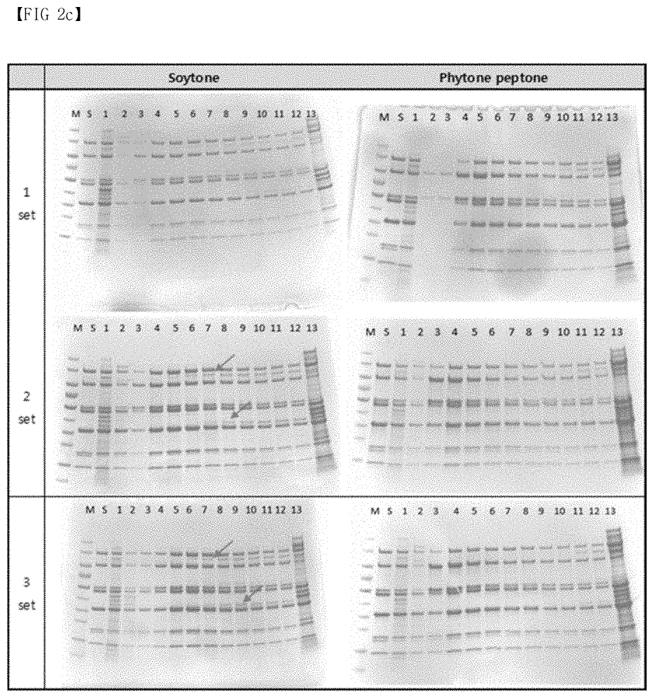

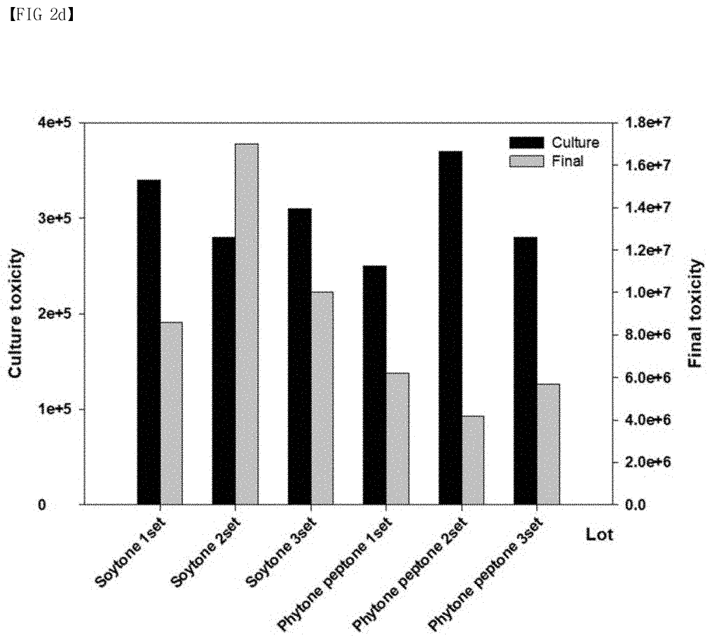

According to another embodiment of the present invention, as a result of isolating botulinum toxin by a process modified from the process shown in FIG. 2A, in which a nucleic acid removal process through protamine sulfate treatment is omitted, a DEAE-Sepharose column volume condition in primary anion-exchange chromatography is changed, and cation-exchange chromatography using a HS-column is added, it was confirmed that the increase in DEAE-Sepharose column volume, even without protamine sulfate treatment, resulted in the same nucleic acid removal effect, and protein concentration was increased about 2 times or more by the addition of the cation-exchange chromatography process (see Example 3).

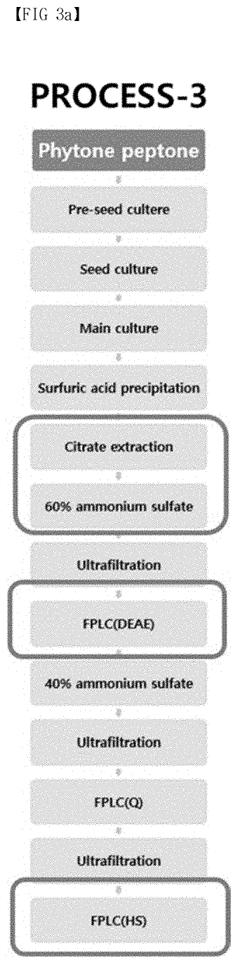

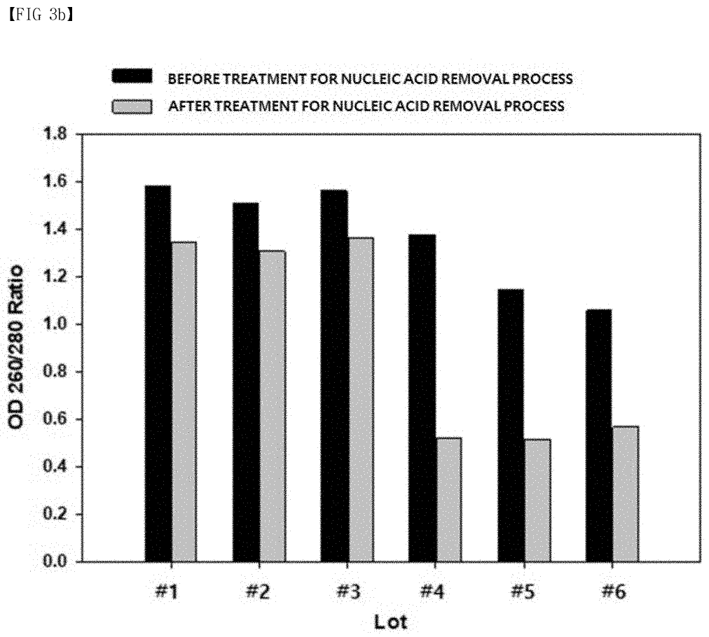

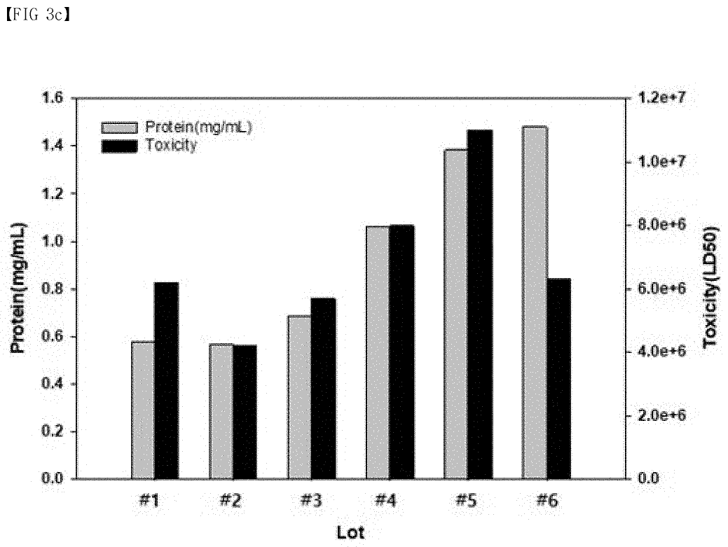

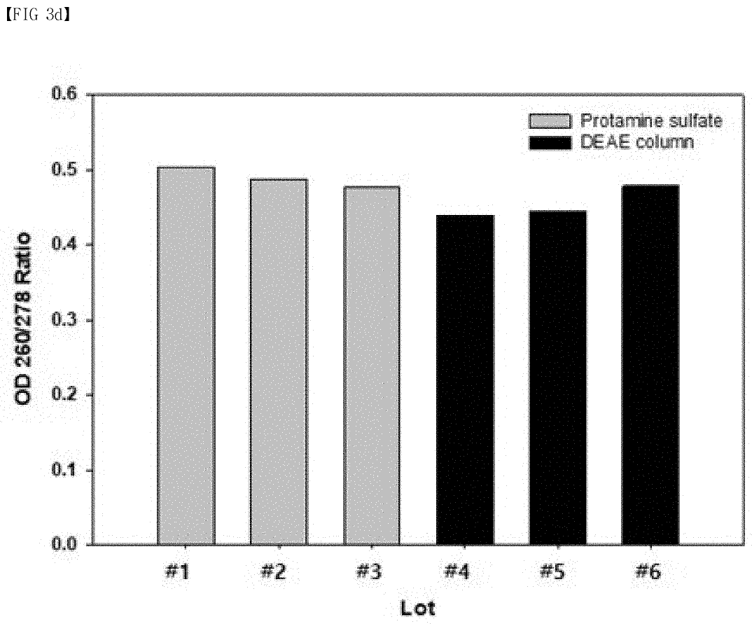

According to still another embodiment of the present invention, as a result of isolating botulinum toxin by a process modified from the process shown in FIG. 3A, in which a Q-Sepharose column process in secondary anion-exchange chromatography is modified, and a buffer used in a HS-column process of cation-exchange chromatography is altered to be the same as that used in the Q-Sepharose column process, it was confirmed that the yield of botulinum toxin protein was increased about 3 times by the modified conditions (see Example 4).

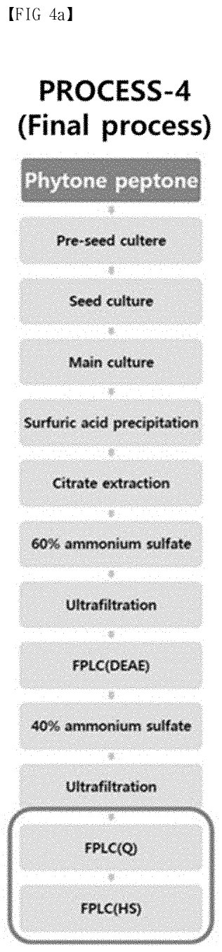

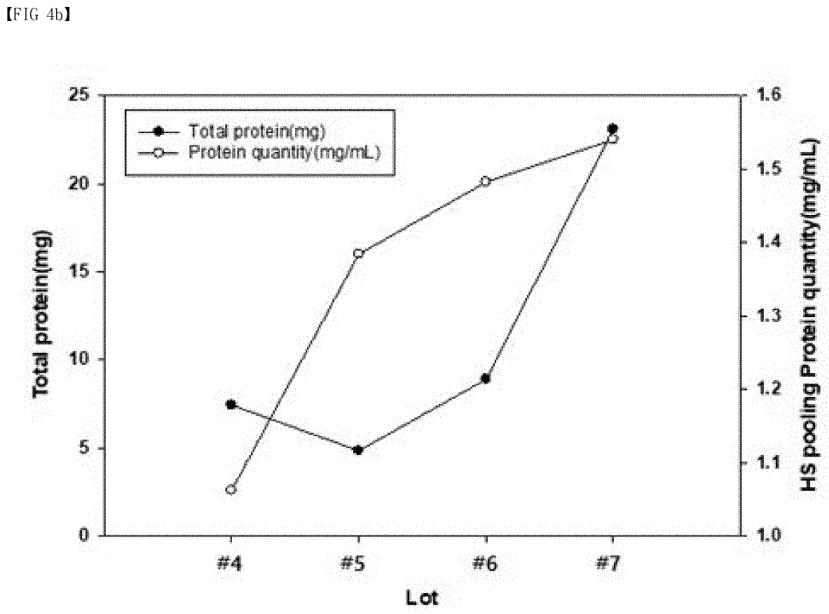

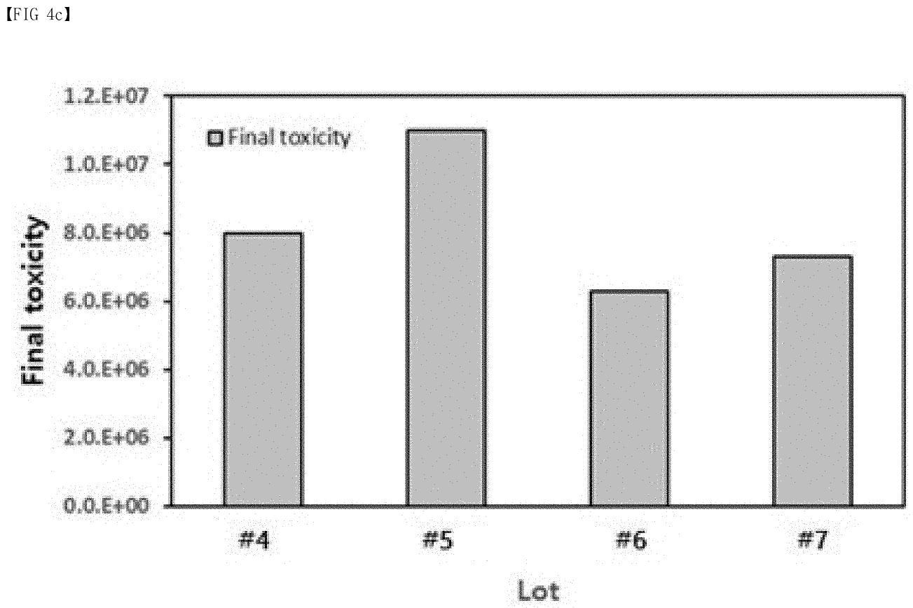

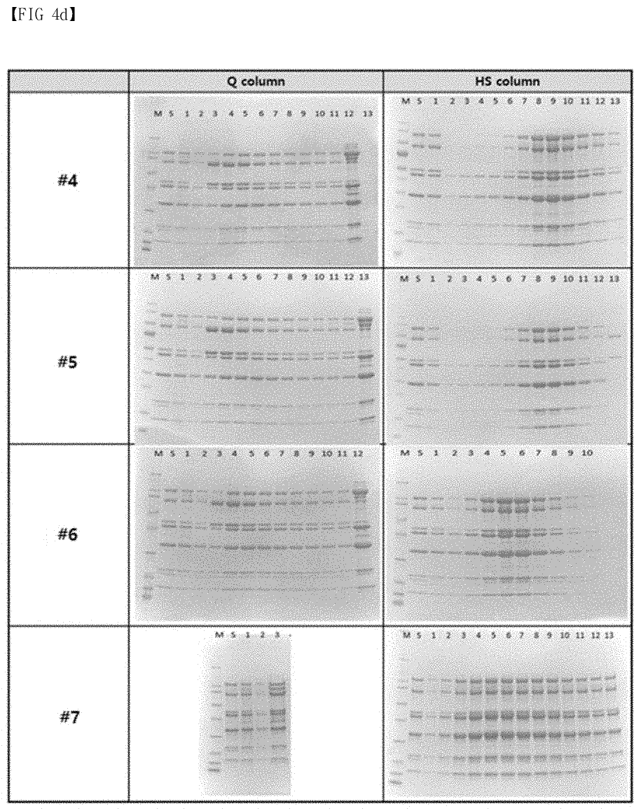

Therefore, from the results of the examples, a process shown in FIG. 4A is established as a final process of preparing botulinum toxin.

Therefore, the present invention provides a method for preparing botulinum toxin, which includes: the steps of:
(a) culturing Clostridium botulinum in a culture medium free of animal-derived components to produce botulinum toxin;
(b) acid-precipitating a liquid culture containing the botulinum toxin produced therein;
(c) adding a buffer to the botulinum toxin-containing precipitate resulting from the step (b) to obtain a supernatant, adding ammonium sulfate to obtain a precipitation supernatant, and performing ultrafiltration;
(d) performing primary anion-exchange chromatography to obtain purified botulinum toxin;
(e) adding ammonium sulfate to the purified botulinum toxin resulting from the step (d) to obtain a precipitation supernatant and performing ultrafiltration;
(f) performing secondary anion-exchange chromatography to obtain purified botulinum toxin; and
(g) performing cation-exchange chromatography to concentrate botulinum toxin.

Hereinafter, the preparation method will be described in detail.

In the present invention, a strain for producing botulinum toxin is preferably Clostridium botulinum or a variant thereof, but the present invention is not limited thereto, and any strain capable of producing botulinum toxin may be appropriately selected and used by those skilled in the art.

“Botulinum toxin” according to the present invention may include not only a neurotoxin (NTX) produced by a Clostridium botulinum strain or a variant thereof but also modified, recombinant, hybrid, and chimeric botulinum toxin. Recombinant botulinum toxin may have light and/or heavy chains recombinantly produced by a non-Clostridium species.

In the present invention, the botulinum toxin may be selected from the group consisting of serotypes A, B, C, D, E, F, and G and include not only pure botulinum toxin (150 kDa) but also botulinum toxin complexes of various sizes (300, 450, 900 kDa).

In the step (a) of the present invention, the culturing of a Clostridium botulinum strain may be performed by appropriate selection and changes by those skilled in the art through a typical method known in the art.

In the culturing, the culture medium is characterized by not containing an animal component and preferably contains phytone peptone, a yeast extract, and glucose which are vegetable components. The culturing may be performed at 25° C. to 40° C. for 72 to 150 hours, more preferably at 30° C. to 38° C. for 90 to 120 hours, and most preferably at 35° C. for 96 hours.

In the step (b) of the present invention, the acid precipitation may be performed by treating a liquid culture containing the botulinum toxin obtained in the step (a) with sulfuric acid or hydrochloric acid, and preferably, sulfuric acid so that a pH becomes pH 3.0 to pH 4.5, preferably pH 3.2 to pH 4.0, more preferably pH 3.3 to pH 3.6, and most preferably pH 3.4.

The acid precipitation kills all of the botulinum strains remaining in the liquid culture and uses the principle that the protein reaches an isoelectric point and precipitates by lowering a pH by adding an acid to various types of protein solutions. In this case, it is known that a lower pH increases the recovery rate of botulinum toxin, but when a pH is 3.0 or less, the botulinum toxin itself is affected, and when the pH is 4.5 or more, the recovery rate of the toxin is lowered. Therefore, the pH range according to the present invention is most appropriate.

In the step (c) of the present invention, the buffer may be sodium citrate with pH 4.5 to pH 6.5, and preferably, pH 5.5, but the present invention is not limited thereto, and any buffer capable of dissolving and extracting the protein pellet precipitated in the step (b) may be appropriately selected and used by those skilled in the art.

The ammonium sulfate precipitation in the step (c) may be performed by slowly adding 40% to 80%(w/v) ammonium sulfate, preferably 50% to 70%(w/v), more preferably 55% to 65%(w/v), and most preferably 60%(w/v) ammonium sulfate to a supernatant obtained by adding the buffer while stirring. The resulting solution may be stored overnight with stirring and then centrifuged to obtain a pellet, and the pellet may be dissolved in a buffer to obtain an ammonium sulfate precipitation supernatant. Afterward, the ammonium sulfate precipitation supernatant is subjected to ultrafiltration, and the buffer may be replaced by 10 times the volume of the ammonium sulfate precipitation supernatant.

As used herein, the term “ultrafiltration” is a process of fractionating a target solute (e.g., botulinum toxin) through the pores of a membrane under a certain pressure according to the size and structure of the solute which is a component of the mixed solution, and is preferably used to separate particles with a size of 0.01 to 0.1 μm. Generally, it is used to remove proteins, endotoxins, viruses, silica, and the like, thereby removing impurities included in the botulinum toxin precipitation liquid and concentrating botulinum toxin.

In the present invention, the steps (d) to (g) are processes for purifying and concentrating botulinum toxin with high purity. To distinguish the anion-exchange chromatography processes, the process of the step (d) is set as primary anion-exchange chromatography, and the process of the step (f) is set as secondary anion-exchange chromatography.

In the step (d), the primary anion-exchange chromatography is preferably performed using a diethylaminoethyl (DEAE)-Sepharose column, and the DEAE-column may have a packing volume of 150 mL to 250 mL, more preferably 180 mL to 220 mL, and even more preferably 200 mL.

The inventors of the present invention have confirmed that, when the packing volume of the DEAE-column is increased from a conventionally used volume (30 to 50 mL) to about 200 mL, an equal level of nucleic acid removal ability is exhibited despite the omission of a protamine sulfate treatment process which is a separate nucleic acid removal process prior to the ammonium sulfate treatment of the step (c).

As the column buffer of the primary anion-exchange chromatography, sodium citrate may be used, but the present invention is not limited thereto. The buffer may have a concentration of 20 to 70 mM, more preferably 40 to 60 mM, and most preferably 50 mM. The buffer may have a pH of 2 to 9, preferably a pH of 3 to 8, more preferably a pH of 4 to 7, and most preferably a pH of 5.5.

As used herein, the term “pH” is a numerical value indicating the degree of acidity or alkalinity of a solution and is an index indicating the concentration of hydrogen ions. Within the range of pH 0 to pH 14, solutions with a pH of 7 are neutral, solutions with a pH of less than 7 are acidic, and solutions with a pH of more than 7 are alkaline. The pH may be measured using a pH meter, and the pH of the buffer may be adjusted using an acid or base such as HCl or NaOH.

As used herein, the term “conductivity” means the ability of an aqueous solution to conduct a current between two electrodes, and since a current flows by ion transport in the solution, conductivity may be controlled by changing the amount of ions present in the aqueous solution. For example, the concentration of a buffer and/or a salt (e.g., sodium chloride, sodium acetate, or potassium chloride) in a solution may be changed to achieve desired conductivity. Preferably, the concentration of salts in various types of buffers may be changed to achieve desired conductivity.

The step (e) of the present invention may be performed by adding ammonium sulfate to the purified botulinum toxin obtained in the step (d) and performing ultrafiltration in the same manner as in the step (c). In this case, the ammonium sulfate may be added so that a concentration becomes 30% to 50%(w/v), more preferably 35% to 45%(w/v), and most preferably 40%(w/v).

The secondary anion-exchange chromatography in the step (f) of the present invention is preferably performed using a Q-Sepharose column, and the buffer may have a pH of 2 to 9, preferably a pH of 3 to 8, more preferably a pH of 4 to 7, and most preferably a pH of 5.5. The botulinum toxin in the step (f) may be obtained as a botulinum toxin-containing fraction from a flow through (FT) eluted from anion-exchange chromatography.

As used herein, the term “flow-through (FT)” process means an isolation method in which, when at least one target molecule (e.g., botulinum toxin) contained along with one or more impurities in a biological product passes through a substance that binds to the one or more impurities, the target molecule does not bind to (that is, flows through) the substance. In the present invention, this is a method of isolating a purified product containing botulinum toxin from a substance binding to a resin of anion-exchange chromatography in the secondary anion-exchange chromatography, and it was confirmed that the yield of botulinum toxin was increased about 3 times or more by using a method of obtaining botulinum toxin as a botulinum toxin-containing fraction from a FT eluted from anion-exchange chromatography.

The cation-exchange chromatography in the step (g) of the present invention is preferably performed using a HS-column, and the buffer may have a pH of 2 to 9, preferably a pH of 3 to 8, more preferably a pH of 4 to 7, and most preferably a pH of 5.5.

In the present invention, as the buffers in the chromatography processes using a Q-Sepharose column and a HS-column, the same sodium citrate is preferably used, and the simplification of the process and an increase in yield of botulinum toxin may be achieved by changing the buffer in the conventional Q-Sepharose column process to be the same as the buffer in the HS-column process and adjusting the concentration thereof.

Hereinafter, exemplary examples will be described for promoting understanding of the present invention. However, the following examples should be considered in a descriptive sense only, and the scope of the present invention is not limited to the examples.

### EXAMPLES

### Example 1

### Basic Process (Process 1)

In order to establish a final process capable of isolating a toxic protein from a Clostridium botulinum strain with excellent efficiency, the inventors of the present invention isolated a toxin by omitting, adding, and changing some processes of the following basic process and results thereof were compared. A basic botulinum toxin isolation process of the present invention is as follows, and each step is simply shown in FIG. 1.

### 1.1 Culture of Strain

First, to perform a pre-seed culture process, 6.25 g of a cooked meat medium (CMM; BD, Cat. 226730) was input into a 100 ml vessel, 50 ml of tertiary distilled water was input, and then sterilization was performed using an autoclave at 122° C. for 30 minutes. After the completion of the sterilization, the resulting vessel was transferred to a biological safety cabinet (BSC), the medium was cooled to 35±2° C., a Clostridium botulinum strain stock was activated in a 35° C. incubator for about an hour during the cooling of the medium and then placed inside the BSC, and 2.5 ml of the stock was inoculated in 50 ml of the CMM (inoculation amount: 5%). Afterward, an anaerobic gas pack, an anaerobic indicator, and the inoculated liquid culture were input into an anaerobic jar, the jar was sealed, and then culture was performed in a 35° C. incubator for 24±2 hours.

Next, to perform a seed culture process, 24 g (3%) of soytone (BD, Cat. No 212488) or phytone peptone (BD, 211906) and 16 g (2%) of a yeast extract (BD, Cat. 212750) were added to tertiary distilled water so that a volume became 700 ml, and the resultant was input into a 1 L vessel. 8 g (1%) of glucose (Merck, Cat. 1.37048.5000) was adjusted to a volume of 100 ml using tertiary distilled water and then input into a separate 150 ml vessel. Then, sterilization was performed using an autoclave at 122° C. for 30 minutes, the resulting vessel was transferred to a BSC, and the medium was cooled to 55 to 60° C. After the cooling of the medium, 100 ml of the glucose was added to 700 ml of the pre-culture medium using a pipette aid. Afterward, when a temperature of the pre-culture medium reached 35±2° C., 16 ml of the 50 ml CMM liquid culture inoculated the previous day was inoculated on the bottom of the vessel (inoculation amount: 2%). Then, an anaerobic gas pack, an anaerobic indicator, and the inoculated liquid culture were input into an anaerobic jar, the jar was sealed, and then culture was performed in a 35° C. incubator for 24±2 hours.

After the completion of the 24-hour culture, to perform a main culture process, 200 g (2%) of soytone or phytone peptone and 100 g (1%) of a yeast extract were input into a 10 L beaker, 8 L of tertiary distilled water was added, and stirring was performed. After the medium composition was completely dissolved, the volume was adjusted to 10 L using tertiary distilled water and then divided into 1.85 L aliquots in 2 L vessels. 60 g (0.5%) of glucose was adjusted to a volume of 300 ml using tertiary distilled water and then input into a separate 500 ml vessel. Then, sterilization was performed at 122° C. for 40 minutes, the resulting vessel was transferred to a BSC, the medium was cooled to 55 to 60° C., and 50 ml of the glucose was added to 1.85 L of the main culture medium that had been divided into the 2 L vessel using a pipette aid (1.9 L of 2 L bottle main culture medium). Afterward, when a temperature of the main culture medium reached 35±2° C., 100 ml of the 800 ml TPM liquid culture inoculated the previous day was inoculated on the bottom of the vessel (inoculation amount: 5%), the vessel was then sealed by tightly closing a lid, and stationary culture was performed in a 35° C. incubator for 96±2 hours.

### 1-2. Sulfuric Acid Precipitation

After the culture was performed according to the method of Example 1-1, a magnetic bar was input into five 2 L vessels where the culture had been completed, the gas was released with stirring, and 3 N sulfuric acid was input to adjust a pH to 3.2 to 3.5. When a pH of 3.2 to 3.5 was reached, the vessel was sealed with a lid and then stored in a 4° C. refrigerator for 12 to 24 hours.

### 1-3. Citrate Buffer Extraction

After the sulfuric acid precipitation was performed for 24 hours according to the method of Example 1-2, the clear upper layer was removed using a pipette aid, and only the precipitate present in the lower layer was centrifuged at 12,000 g for 30 minutes. Afterward, a supernatant was discarded to collect only a pellet, 500 ml of 200 mM sodium citrate (pH 5.5; Merck, Cat. 1.37042.5000) was added to suspend the pellet, and then the suspension was stirred in a 4° C. refrigerator for an hour. After the completion of the stirring, primary centrifugation was performed at 12,000 g for 30 minutes, a supernatant was separately stored in a 1 L vessel (4° C.), and a pellet was suspended with 500 ml of the same 200 mM sodium citrate solution. Then, secondary centrifugation was performed in the same manner, and a supernatant was combined with the supernatant obtained by the primary centrifugation, thereby obtaining a sulfuric acid precipitation extraction supernatant.

### 1-4. Protamine Sulfate Treatment

A 2% protamine sulfate solution (Merck, Cat. 1.10123.0025) was prepared in advance during the preceding steps and then slowly dropped using a separatory funnel so that a volume reached 0.1% of the volume of the obtained supernatant (treatment with 50 ml of the 2% protamine sulfate solution based on 1 L of the volume of the obtained supernatant). Afterward, stirring was performed at room temperature for 20 minutes, and centrifugation was performed at 12,000 g for 30 minutes, thereby obtaining a supernatant.

### 1-5. Ammonium Sulfate Precipitation

Ammonium sulfate (60%(w/v), 36.1 g based on 100 ml) (Merck, Cat. 1.01816.5000) was slowly added to the supernatant obtained in Example 1-4 with stirring and then stirred at 4° C. overnight using a stirrer. Afterward, centrifugation was performed at 12,000 g for 30 minutes to obtain a precipitate, and the precipitate was dissolved in 100 ml of a 50 mM sodium citrate buffer (pH 5.5), thereby obtaining an ammonium sulfate precipitation supernatant.

### 1-6. Replacement of Buffer Through Ultrafiltration (UF)

The obtained ammonium sulfate precipitation supernatant was input, and the pump speed of a UF system was set to 2 gauge. The 50 mM sodium citrate (pH 5.5) solution to be replaced was replaced with a 10-fold volume of the ammonium sulfate precipitation supernatant. In this case, care was taken that the pump inlet pressure did not exceed 2 bar, and the recovered concentrate was stored at 4° C.

### 1-7. Chromatography Purification Using DEAE Column

It was attempted to purify a toxin from the obtained concentrate through an anion-exchange chromatography method using a DEAE column For this purpose, first, 50 mM sodium citrate (pH 5.5) as a running buffer and a solution of 50 mM sodium citrate (pH 5.5) and 1 M sodium chloride (pH 5.5) (Merck, Cat. 1.06400.5000) as an elution buffer were prepared, filtered through a 0.22 μm filter, and then sonicated to remove air. Subsequently, after turning on a fast protein liquid chromatography (FPLC) device and washing the pump with the running buffer, a pump A1 was immersed in the running buffer, a pump B1 was immersed in a sample buffer and the elution buffer, and a sample A1 loop was immersed in a purification sample, and the DEAE column was equilibrated using the DEAE column washing method (elution buffer 5CV, running buffer 5CV). Afterwards, purification was performed using the DEAE column method as follows: \textcircled{1} equilibration (running buffer 1CV), \textcircled{2} sample application, \textcircled{3} column washing (running buffer 2CV), \textcircled{4} column washing (elution buffer 2CV). After the completion of the purification, the DEAE column was washed using a pump A1 as follows: \textcircled{1} 0.5 N NaOH, 5 mL/min, 2CV, \textcircled{2} D.W, 5 mL/min, until conductivity was stabilized, \textcircled{3} running buffer, 5 mL/min, until pH was stabilized, \textcircled{4} 20% ethanol (EtOH), 5 mL/min, 3CV. After the completion of the column washing, all of the loops were immersed in 20% ethanol to wash the pump, and then the washing was terminated. The purified fraction was sampled by pooling only the desired fraction after confirming the protein through SDS-PAGE. Afterward, ammonium sulfate (40%(w/v), 22.6 g based on 100 ml) was slowly added to the purified liquid with stirring and then stored at 4° C. overnight (24 hours). Subsequently, the purified liquid obtained by the above method was subjected to a buffer replacement process through ultrafiltration (UF) in the same manner as in Example 1-6 to recover a concentrate, and then purification using a Q-column was performed according to the following method.

### 1-8. Chromatography Purification Using Q Column

It was attempted to purify a toxin from the obtained concentrate through an ion-exchange chromatography method using a Q column. For this purpose, first, 20 mM sodium phosphate (pH 6.5) as a running buffer and a solution of 20 mM sodium phosphate (pH 6.5) and 1 M sodium chloride (pH 6.5) as an elution buffer were prepared, filtered through a 0.22 pm filter, and then sonicated to remove air. Subsequently, after turning on the FPLC device and washing the pump with the running buffer, a pump A1 was immersed in the running buffer, a pump B1 was immersed in a sample buffer and the elution buffer, and a sample A1 loop was immersed in a purification sample, and the Q column was equilibrated using the Q column washing method (elution buffer SCV, running buffer 5CV). Afterwards, purification was performed using the Q column method as follows: \textcircled{1} equilibration (running buffer 1CV), \textcircled{2} sample application, \textcircled{3} column washing (running buffer 2CV), \textcircled{4} column washing (elution buffer 5, 15, 50, 100% each 2CV). After the completion of the purification, the Q column was washed using a pump A1 as follows: \textcircled{1} 0.5 N NaOH, 5 mL/min, 2CV, \textcircled{2} D.W, 5 mL/min, until conductivity was stabilized, \textcircled{3} running buffer, 5 mL/min, until pH was stabilized, \textcircled{4} 20% ethanol (EtOH), 5 mL/min, 3CV. After the completion of the column washing, all of the loops were immersed in 20% ethanol to wash the pump, and then the washing was terminated. The purified fraction was sampled by pooling only the desired fraction after confirming the protein through SDS-PAGE.

The buffer conductivity, conductivity before buffer replacement, and conductivity after buffer replacement of the buffers applied to the DEAE-column and Q column in Example 1-7 and Example 1-8 were measured, and results thereof were shown in the following Table 1.

TABLE 1

| | Buffer | Conductivity before | Conductivity after |
|---|---|---|---|
| | conductivity | buffer replacement | buffer replacement |
| Process | (mS/cm) | (mS/cm) | (mS/cm) |
| DEAE column | 10.747 | 74.940 | 11.800 |
| Q column | 2.704 | 8.105 | 2.928 |

### Example 2

### Comparison of Effect According to Change in Medium Component and Column Volume Conditions (Process 2)

The inventors of the present invention attempted to establish an optimum process with high toxin production yield by changing conditions of some steps of the basic process of Example 1. For this purpose, first, botulinum toxin was isolated as shown in FIG. 2A by changing conditions of a vegetable medium component and the Q column purification of Example 1-8 in the Process 1, and results thereof were compared.

More specifically, as shown in the following Table 2, a vegetable medium component was changed from soytone to phytone peptone, the main culture was performed for 96 hours or 72 hours as in the basic process, the packing volume of a DEAE-Sepharose column in the purification process was changed from 30 mL to 200 mL, and the packing volume of a Q-Sepharose column was increased from 30 mL to 50 mL, which was intended to increase a binding capacity.

TABLE 2

| | | | | | | | Phytone peptone | | | | | |
|---|---|---|---|---|---|---|---|---|---|---|---|---|
| | Soytone | | | | | | 1 set | | 2 set | | 3 set | |
| | 1 set | | 2 set | | 3 set | | (Process 2) | | (Process 3) | | (Process 3) | |
| Culture time | 96 | h | 96 | h | 72 | h | 96 | h | 96 | h | 96 | h |
| DEAE vol. | 30 | mL | 30 | mL | 30 | mL | 30 | mL | 200 | mL | 200 | mL |
| Q vol. | 30 | mL | 30 | mL | 30 | mL | 50 | mL | 50 | mL | 50 | mL |

The botulinum toxin protein was isolated according to the process of FIG. 2A by applying each changed condition as shown in Table 2, and the protein concentration for each lot, the SDS-PAGE result of the protein fraction after Q purification, and the toxicity of the toxic protein for each lot were compared. As a result, as shown in FIG. 2B and the following Table 3, it was confirmed that the protein concentration (mg/mL; black bar) of the final purified liquid in the case of culture using a phytone peptone medium was lower than that in the case of culture using a soytone medium by about half, but considering a volume difference, the total protein amount (mg; gray bar) was measured to be higher when a phytone peptone medium was used.

TABLE 3

| | | | | Phytone peptone | | |
|---|---|---|---|---|---|---|
| | Soytone | | | 1 set | 2 set | 3 set |
| | 1 set | 2 set | 3 set | (Process 2) | (Process 3) | (Process 3) |
| Protein (mg/mL) | 0.794 | 1.094 | 1.137 | 0.576 | 0.568 | 0.684 |
| Protein (mg) | 8.734 | 8.095 | 12.507 | 19.584 | 11.360 | 23.940 |
| Pooling vol (mL) | 11 | 7.4 | 11 | 34 | 20 | 35 |

In addition, the purified liquid obtained after a chromatography purification process using a Q column was subjected to SDS-polyacrylamide gel electrophoresis (PAGE) to confirm protein bands. As a result, as shown in FIG. 2C, protein bands (red arrow) for impurities were not observed in the case of culture in a phytone peptone medium unlike the result of a soytone medium.

Finally, as a result of measuring the toxicity of the botulinum toxin protein in the culture supernatant and final purified liquid after the main culture, as shown in FIG. 2D and the following Table 4, the toxicity of the culture supernatants was all at a similar level. However, the toxicity of the final purified liquid was about 2 to 3 times higher in the case of culture using a soytone medium, and this was considered to be due to a decrease in protein concentration as the pooling volume of the fraction increased about 3 times after Q purification.

TABLE 4

| | | | | Phytone peptone | | |
|---|---|---|---|---|---|---|
| | Soytone | | | 1 set | 2 set | 3 set |
| | 1 set | 2 set | 3 set | (Process 2) | (Process 3) | (Process 3) |
| Culture | 3.4*10⁵ | 2.8*10⁵ | 3.1*10⁵ | 2.5*10⁵ | 3.7*10⁵ | 2.8*10⁵ |
| supernatant | or more | | | | | |
| Final purified | 8.6*10⁶ | 1.7*10⁷ | 1.0*10⁷ | 6.2*10⁶ | 4.2*10⁶ | 5.7*10⁶ |
| liquid | | | | | | |

Referring to the results of Example 2, a vegetable medium component was changed from soytone to the final phytone peptone, and the packing volume of a Q-Sepharose column was increased from 30 mL to 50 mL to increase a recovery rate.

### Example 3

### Comparison of Effect According to Deletion of Protamine Sulfate Treatment Process and Addition of Purification Process (Process 3)

The inventors of the present invention purified a botulinum toxin protein by a process shown in FIG. 3A, in which conditions of a nucleic acid removal process using protamine sulfate and a chromatography purification process were changed from the Process 2 in which a vegetable medium component and the packing volume of a Q-Sepharose column were changed, and then compared the effects thereof.

More specifically, the packing volume of a DEAE-Sepharose column was changed from 30 mL to 200 mL, and the nucleic acid removal rate before and after protamine sulfate treatment and the nucleic acid removal rate before and after DEAE-Sepharose column treatment were compared. In this case, lots #1, #2, and #3 in the following Table 5 proceeded under conditions of the soytone media 1 set, 2 set, and 3 set shown in Table 2 of Example 2, and lots #4, #5, and #6 in the following Table 5 proceeded under the condition of repeating the 3 sets of the phytone peptone medium shown in Table 2 of Example 2. Also, to compare effects before and after a nucleic acid removal process, the nucleic acid removal efficiency before and after protamine sulfate treatment and the nucleic acid removal efficiency before and after DEAE-Sepharose column treatment after changing the packing volume of the DEAE-Sepharose column from 30 mL to 200 mL were measured.

TABLE 5

| | | OD260/278 ratio | OD260/278 ratio |
|---|---|---|---|
| Nucleic acid removal | | before nucleic acid | after nucleic acid |
| process | Lot | removal process | removal process |
| Protamine sulfate | #1 | 1.585 | 1.349 |
| | #2 | 1.507 | 1.309 |
| | #3 | 1.564 | 1.366 |
| DEAE column work | #4 | 1.377 | 0.522 |
| | #5 | 1.144 | 0.518 |
| | #6 | 1.059 | 0.570 |

As a result, as shown in FIG. 3B and Table 5, it was confirmed that OD260/278 ratio values after a nucleic acid removal process were lower than OD260/278 ratio values before a nucleic acid removal process, and thus the nucleic acid removal effect was exhibited in both the results before and after protaminesulfate treatment (#1, #2, #3) and the results before and after DEAE-Sepharose column treatment (#4, #5, #6). This is the result of confirming that nucleic acid removal is possible by increasing the volume of a DEAE-Sepharose column even without a protamine sulfate treatment process.

In addition, the inventors of the present invention added a concentration process using a HS-column which is an ion-exchange column and compared a concentration result with that in the case of Process 2. As a result, as shown in FIG. 3C, it was confirmed that the protein concentrations (mg/mL) in #4 (1.062 mg), #5 (1.384 mg), and #6 (1.482 mg), which used a HS-column, were about 2 times higher than those in #1 (0.576 mg), #2 (0.568 mg), and #3 (0.684 mg) which used a Q-column. This shows that the degree of concentration is significantly improved when HS-column purification is added, indicating that it is possible to perform a purity test to check impurities.

Additionally, as a result of performing the final purification of Process 3 in which a HS-column process was added and measuring the toxicity of a botulinum toxin protein in the final purified liquid, as shown in FIG. 3C, it was confirmed that the toxicities in #4, #5, and #6, which used a HS-column, were higher than those in #1, #2, and #which used a Q-column

Finally, the inventors of the present invention compared the nucleic acid removal efficiency (#1, #2, #3) of the final purified liquid that had been subjected to the removal of nucleic acid by a protamine sulfate treatment process and Q-column purification and the nucleic acid removal efficiency (#4, #5, #6) of the final purified liquid that had been subjected to the removal of nucleic acid by a DEAE-Sepharose column treatment process and HS-column purification according to Process 3, as shown in the following Table 6.

TABLE 6

| Nucleic acid | | | Final | |
|---|---|---|---|---|
| removal | | | OD260/278 | |
| process | Lot | Final step | ratio | Average |
| Protamine | #1 | Q column purification | 0.504 | 0.490 |
| sulfate | | Pooling | | |
| | #2 | Q column purification | 0.488 | |
| | | Pooling | | |
| | #3 | Q column purification | 0.477 | |
| | | Pooling | | |
| DEAE column | #4 | HS column purification | 0.439 | 0.454 |
| | | Pooling | | |
| | #5 | HS column purification | 0.445 | |
| | | Pooling | | |
| | #6 | HS column purification | 0.479 | |
| | | Pooling | | |

As a result, as shown in FIG. 3D and Table 6, there was no significant difference between the nucleic acid removal efficiency (gray bar) of the final purified liquid that had been subjected to the removal of nucleic acid by protamine sulfate treatment and a Q-column purification process and the nucleic acid removal efficiency (black bar) of the final purified liquid that had been subjected to the removal of nucleic acid by a DEAE-Sepharose column and a HS-column purification process. Therefore, it was confirmed that, when the process proceeded by selecting a DEAE column instead of protamine sulfate in nucleic acid removal and changing the packing volume of a DEAE-Sepharose column from 30 mL to 200 mL, there was no significant difference in nucleic acid content in the final product from Process 2, and an equal level of nucleic acid removal ability was exhibited. Referring to the results of Example 3, the packing volume of a DEAE-Sepharose column was changed from 30 mL to 200 mL, the protamine sulfate treatment process of Process 2 was deleted, and a purification process using a HS-column was added to solve the problem of concentration dilution.

### Example 4

### Comparison of Effect According to Change in Condition of Chromatography Purification Process (Process 4, Final Process)

The inventors of the present invention isolated a botulinum toxin protein according to a process shown in FIG. 4A by changing some of the chromatography process conditions from Process 3 of Example 3 and compared the effect with the effect of Process 3.

More specifically, when compared with Process 3, Process 4 uses a method of recovering a protein in a flow-through (FT) manner without binding to a resin instead of a method of eluting a protein by binding to a resin in chromatography using a Q-Sepharose column, and the buffers used in Q-Sepharose and HS column processes were changed to be the same 10 mM sodium citrate (pH 5.5). Also, to solve the problem in purification using a DEAE column, the buffer replacement process after treatment with 60% ammonium sulfate described in Example 1-6 was performed by a method using a dialysis tube instead of an existing ultrafiltration method. Comparative groups according to the changed conditions are summarized in Table 7 below, #4, #5, and #6 correspond to the case of Process 3 in which a buffer replacement process varies, and #7 correspond to the case of Process 4 in which the changed conditions are applied. The botulinum toxin protein was isolated by the process according to each of the changed conditions, and then the protein concentration and toxicity for each lot and the SDS-PAGE result of the purified liquid were compared.

First, as a result of comparatively analyzing the concentration of protein in the final purified liquid, as shown in FIG. 4B and Table 7 below, it was confirmed that the protein yield in #7 (23.100 mg) corresponding to Process 4 was increased about 3 times as compared with that in #4 (7.430 mg), #5 (4.844 mg), and #6 (8.892 mg) corresponding to Process 3. This shows that the protein yield is significantly improved by the conditions for recovery in a flow-through manner in Q-column purification and changing the buffers used in Q-Sepharose and HS column processes to be the same.

In addition, as a result of analyzing the toxicity of the final purified liquid, as shown in FIG. 4C and the following Table 7, there was no significant difference in protein toxicity according to a process difference.

TABLE 7

| Lot | #4 | #5 | #6 | #7 |
|---|---|---|---|---|
| Protein (mg/mL) | 1.062 | 1.384 | 1.482 | 1.540 |
| Pooling vol (mL) | 7.0 | 3.5 | 6.0 | 15.0 |
| Protein (mg) | 7.430 | 4.844 | 8.892 | 23.100 |
| LD50 | 8.0*10⁶ | 1.1*10⁷ | 6.3*10⁶ | 7.3*10⁶ |

Additionally, as a result of subjecting the purified liquid to SDS-PAGE after Q-column purification and after HS-column purification, as shown in FIG. 4D, protein bands for impurities were not observed in all of the cases.

The aforementioned description of the present invention is provided by way of example and those skilled in the art will understood that the present invention can be easily changed or modified into other specified forms without change or modification of the technical spirit or essential characteristics of the present invention. Therefore, it should be understood that the aforementioned examples are only provided by way of example and not provided to limit the present invention.

### INDUSTRIAL APPLICABILITY

It was confirmed that the method for preparing botulinum toxin according to the present invention provides excellent safety and is capable of isolating botulinum toxin with significantly improved yield, and thus the method is expected to be usefully used in beauty and medicine fields.

1. A method for preparing botulinum toxin, comprising the following steps of:
(a) culturing Clostridium botulinum in a culture medium free of animal-derived components to produce botulinum toxin;
(b) acid-precipitating a liquid culture containing the botulinum toxin produced therein;
(c) adding a buffer to the botulinum toxin-containing precipitate resulting from the step (b) to obtain a supernatant, adding ammonium sulfate to obtain a precipitation supernatant, and performing ultrafiltration;
(d) performing primary anion-exchange chromatography to obtain purified botulinum toxin;
(e) adding ammonium sulfate to the purified botulinum toxin resulting from the step (d) to obtain a precipitation supernatant and performing ultrafiltration;
(f) performing secondary anion-exchange chromatography to obtain purified botulinum toxin; and
(g) performing cation-exchange chromatography to concentrate botulinum toxin.

2. The method of claim 1, wherein the culture medium in the step (a) contains phytone peptone, a yeast extract, and glucose.

3. The method of claim 1, wherein the acid precipitation in the step (b) is performed by adding sulfuric acid or hydrochloric acid so that a pH becomes pH 3.0 to pH 4.5.

4. The method of claim 1, wherein the buffer in the step (c) is sodium citrate with pH 4.5 to pH 6.5.

5. The method of claim 1, a separate nucleic acid removal process is omitted before the addition of ammonium sulfate in the step (c).

6. The method of claim 1, wherein the ammonium sulfate in the step (c) is added so that a concentration becomes 40% to 80%(w/v).

7. The method of claim 1, wherein the primary anion-exchange chromatography is performed using a diethylaminoethyl (DEAE)-Sepharose column

8. The method of claim 7, wherein the DEAE-column has a packing volume of 150 mL to 250 mL.

9. The method of claim 1, wherein the secondary anion-exchange chromatography is performed using a Q-Sepharose column.

10. The method of claim 1, wherein the botulinum toxin in the step (f) is obtained as a botulinum toxin-containing fraction from a flow through (FT) eluted from anion-exchange chromatography.

11. The method of claim 1, wherein the cation-exchange chromatography is performed using a HS-column.

12. The method of claim 1, wherein the chromatography processes in the steps (d), (f), and (g) are performed using the same sodium citrate buffer with pH 4.5 to pH 6.5.
